(19) United States
(12) Patent Application Publication
(10) Pub. No.: US 2018/0024064 A1
(43) Pub. Date: Jan. 25, 2018

(54) METHOD OF DETECTING FIBROUS TISSUE IN A BIOLOGICAL SPECIMEN USING CO-LOCALIZED IMAGES GENERATED BY SECOND HARMONIC GENERATION AND TWO PHOTON EMISSION

(71) Applicant: HISTOINDEX PTE LTD, Singapore (SG)

(72) Inventors: Gideon HO, Singapore (SG); Laurent GOLE, Proteos (SG); Weimiao YU, Proteos (SG)

(21) Appl. No.: 15/547,760

(22) PCT Filed: Mar. 18, 2016
(86) PCT No.: PCT/SG2016/050129

§ 371 Date: Jul. 31, 2017

(30) Foreign Application Priority Data
Mar. 19, 2015 (SG) 10201502151S

(51) Int. Cl. G01N 21/64; G06T 7/00; G01N 33/483

(52) U.S. Cl. CPC G01N 21/6486; G01N 33/4833; G06T 7/0012; G01N 2021/6439; G06T 2207/10064; G06T 2207/30024; G06T 7/45

(57) ABSTRACT

The present disclosure offers a method of detecting the presence of fibrous tissue deposited to a biological specimen or tissue sample. Preferably, the method comprises the steps of illuminating the specimen using an electromagnetic radiation of an excitation wavelength that the specimen contains excitable materials or compound to result in auto-fluorescence emitting a first electromagnetic signal caused by two-photon excitation, the specimen contains non-linear materials to generate a second electromagnetic signal in the form of second harmonic wave pursuant to the illuminating step; recording the first and the second electromagnetic signals emitted from the specimen; co-localizing the recorded first and second electromagnetic signals to generate an image; and detecting presence of the fibrous tissue deposited to the specimen using the generated image. More preferably, the light source is a laser beam or the like operable to induce two-photon excitation in the given tissue sample.

### TECHNICAL FIELD

The present disclosure relates to a method of detecting and/or quantifying the level of fibrosis or deposition of collagen fibers in a biological test specimen, preferably acquired from a subject, by exploiting auto-fluorescence property of the endogenous fluorophores and/or optically non-linear materials found in the specimen. The disclosed method further facilitates prognosis or diagnosis of a diseased state based upon the result of the fibrosis and/or deposition of collagen fibers quantified and/or detected. The present disclosure also includes a system implementable for detecting and/or quantifying deposition of collagen fiber and correlating the quantified collagen fiber deposition to one or more stages of at least one predetermined disease.

### BACKGROUND

Auto-fluorescence refers to a phenomenon where the natural emission of light or electromagnetic wave of specific spectrum from a material or structure after the material or structure is being illuminated by a light source and absorbs some energy therefrom. Emission, due to auto-fluorescence, posts consistent problems in the field of fluorescence microscopy that the auto-fluorescence light emitted constantly interferes with signal released by artificial fluorophores, as markers, administrated to a biological sample for identification or recognizing one or more specific cellular structures. Apart from that, attempts have been made to further exploit such phenomenon for developing non-destructive approach for diagnosis of one or more diseased states of a subject. For instance, Banerjee describes a method of detecting cancer cell utilizing cellular auto-fluorescence in United States patent publication no. 2004/0038320. Particularly, Banerjee teaches to detect neoplasia by exposing potential neoplastic test tissue sample to ultraviolet light; measuring auto-fluorescence at a wavelength indicative of tryptophan emission; and determining presence of neoplasia by comparing the intensity of the emission from the test tissue sample to standard references. Provenzano et. al. provide another approach to identifying breast tumor cells in U.S. Pat. No. 8,144,966 that the disclosed method identifies the tumor cells by measuring fluorescence intensities, fluorescence lifetime values or both from endogenous FAD in a tissue sample. Another U.S. Pat. No. 8,234,078 discloses a method of classifying tissue using multimodal spectroscopy that the disclosed method collects spectroscopic response data of an illuminated tissue sample and compares the response data with an empirical equation to determine attributes being at least partially indicative of a tissue type. All the aforementioned methods generally employ high energy and single photon excitation to acquire good fluorescence signal and reading. However, single photon-induced fluorescence tends to cause out-of-focus readings and noise to the collectable signals especially when the radiation penetrates deeper into the tissue. These methods may not be feasible in acquiring useful data from thick tissue sample.

Therefore, addition effort has been put into developing microscopy imaging and detection system working on multi-photon excitation principle, which offers deeper photon penetration in tissue sample and illuminates cellular structures thereto without suffering much scattering of strayed fluorescent. European patent application no. 2979121 and U.S. Pat. No. 7,456,378 details microscopy imaging systems operable on multi-photon excitation fluorescence. Also, there are more advanced models capitalizing on multimodal fluorescent events. Preferably, multiple sensors are used in such systems to register signals emitted associated to the multiple spectroscopic phenomenon triggered in the excited sample yielding more data thereof with respect to the tissue sample. For example, Sun et al. describes in International Patent Publication No. 2008/002278 about a microscopy imaging system capable of using optical emission relating to multi-photon excitation and second harmonic generation in generating microscopy image. Also, Ammar et al.'s disclosure, published in United States patent with publication no. 20130149734, claims an imaging system built to simultaneously detect and record signals emitted from excited tissue sample according to more than one optical phenomenon induced in the tissue including photon excitation fluorescence, two photon autofluorescence, fluorescence lifetime imaging, autofluorescence lifetime imaging, second harmonic generation, third harmonic generation, coherent anti-stokes Raman scattering (CARS), broadband or multiplex CARS, stimulated Raman scattering, stimulated emission, nonlinear absorption, and micro-Raman microscopy. However, most of the abovementioned systems fall short on the analytic tools for mining useful information beneficial for prognostic and/or diagnostic needs despite the sheer amount of data captured in the multimodal systems. Therefore, an imaging system equipped with one or more analytic tools lacking in the aforementioned multimodal systems is greatly desired.

### SUMMARY

The present disclosure aims to provide a method for detecting deposition of fibrous tissue or collagen fibers in a biological or biopsy test specimen. The detection of the fibrous tissue in the given specimen through the present disclosure is rapid and non-destructive compared to reticulin or trichrome staining applied in conventional approach to stain the specimen.

Another object of the present disclosure is to offer a method of detecting or identifying fibrous tissue deposited in a biological specimen by collecting different auto-fluorescence or spectroscopic signals, under separate contrast mechanisms, released from the test specimen upon subjecting the test specimen to an irradiation by a radiation source. The irradiation is succeeded by optical phenomenon such as two-photon excitation (TPE) and second harmonic generation (SHG) occurred in the irradiated test specimen. The disclosed method composes a co-localized image based upon the signals collected from TPE and SHG then subsequently quantified collagen fibers deposited in the test specimen using the composed co-localized image.

Further object of the present disclosure targets to offer a method capable to correlate, diagnose or determine diseased state of a subject through assessing or analyzing the aforesaid co-localized image generated based on a biological test specimen acquired from the subject. Preferably, the correlation or diagnosis is performed with aid of a fuzzy logic to minimalize inter-observer variability present in the conventional methods. More specifically, the disclosed method subjects a co-localized image, created by combining signals of TPE and SHG emitted from the specimen after it is irradiated by an electromagnetic radiation, to a computer-implementable process to carry out the necessary steps to analyze the specimen. The diseased state of the subject, whom the specimen is acquired from, can be diagnosed or predetermined based on the result or outcome of the analysis.

Still, an object of the present disclosure is to disclose an image created by co-localizing the electromagnetic signals released from a biological specimen subjected to two-photon excitation after irradiation of a light source that the electromagnetic signals are emitted concurrently from the cellular structure as a result of TPE and SHG.

Further object of the present disclosure is directed to an image generating and analyzing system operable substantially based upon the aforesaid methods to irradiate a test specimen, capture optical signals emitted from the irradiated specimen, and/or generate a co-localized image using the captured optical signals. The disclosed system includes one or more computing or microprocessor units to run the disclosed method implemented in the form of computer readable code for composing the co-localized image.

More object of the present disclosure is directed to a system being configured to mine information beneficial for deriving a prognostic or diagnostic result with respect to the test specimen. The information mining in the disclosed system is preferably a fully automated process with user adjustable parameters.

For one aspect, the present disclosure is a method of detecting presence of fibrous tissue deposited to a biological specimen or tissue sample comprising the steps of irradiating the specimen under a light source of an excitation wavelength for two-photon excitation that the specimen contains excitable materials or compound to result in auto-fluorescence emitting a first electromagnetic signal, the specimen contains non-linear materials to generate a second electromagnetic signal in the form of second harmonic wave pursuant to the excitation; recording the first and the second electromagnetic signals emitted from the specimen subjected to the two-photons excitation; co-localizing the recorded first and second electromagnetic signals to generate an image; and detecting presence of the fibrous tissue deposited to the specimen using the generated image.

Another aspect of the present disclosure relates to a method of image generating and analyzing for quantifying collagen fibers in a biological test specimen. The disclosed method generally comprises the steps of irradiating an interested region of the test specimen using an electromagnetic light source through an optical assembly at an excitation wavelength, the irradiated test specimen having excitable materials and optically non-linear materials in the collagen fibers respectively leading to concurrent emission of a first electromagnetic signal caused by two-photon excitation (TPE) and a second electromagnetic signal as a result of second harmonic generation (SHG); manipulating the first and second electromagnetic signals emitted from the test specimen through the optical assembly; recording the manipulated first and the second electromagnetic signals through one or more sensors; using the concurrently recorded first and second electromagnetic signals to generate a co-localized image having a set of image properties and being indicative of spatial distribution of the collagen fibers in the interested region; and quantifying one or more scalar features and distribution features of the co-localized image to generate quantified result for each scalar and/or distribution feature, and deposition of collagen fibers in the test specimen using the quantified results for the extracted scalar and distribution features. Preferably, the distribution features of the co-localized image are any one or combinations of intensity and/or distribution of the second electromagnetic signal, intensity and/or distribution of the first electromagnetic signal, gray level co-occurrence matrix (GLCM), thickness of collagen fiber, length of the collagen fibers, orientation of the collagen fibers, and straightness of the collagen fibers. Meanwhile, the scalar features of the co-localized image are any one or combinations of collagen proportion ratio, total average collagen, complexity of collagen fibers, and fragment average ratio of the collagen fibers.

According to some other embodiments, the disclosed method further comprises the step of calculating at least one of mean, variance, skewness, kurtosis, energy and entropy for each of the distribution features. These quantified image properties of the co-localized image are subsequently employed for information mining to conclude the prognostic and/or diagnostic results.

For some embodiment, the disclosed method is configured to derive an absolute total average collagen by way of subjecting the co-localized image to multiple iterations of enhancement that each iteration of enhancement corresponds to a quotient of enhancement including N-fold amplification of the default pixel intensity of the co-localized image, calculating total average collagen for each iteration, generating a graft with the calculated total average collagen plotting against the quotient of enhancement, identifying a inflexion point from the plotted graph, and referring the total average collagen on the graph corresponding to the identified inflexion point to derive the absolute total average collagen. Preferably, N is 0.1 to 100.

To ensure the collagen fibers quantified are deposited in the test specimen purely associated to progression of the diseased state to be determined rather than congenital collagen fibers of the cellular structure, the disclosed method identifies collagen fibers associated to blood vessels of the test specimen generated in the co-localized image and excludes the identified collagen fibers associated to blood vessels in the process of deriving the absolute total average collagen according to a number of the preferred embodiments.

For a plurality of the embodiments, the disclosed method also acquire at least preliminary prognostic or diagnostic result from the generated graph by mapping the generated graph against a standard graph calculated from a non-diseased specimen and deriving a prognosis towards a diseased state associated to the test specimen based upon relative distance between the mapped generated graph and the standard graph. To facilitate rapid derivation of a prognostic result, both generated graph and standard graph are line graph.

Still, in several embodiments, the disclosed method in fact derives a prognosis towards a diseased state associated to the test specimen based upon a distance of the inflexion point of the generated graph in relation to a standard graph calculated from a non-diseased specimen.

For several preferred embodiments, the present disclosure offers a method of evaluating a test biological specimen or tissue sample for determination presence of a diseased state comprising the steps of irradiating the specimen using a light source of an excitation wavelength that the specimen contains excitable materials or compound to result in auto-fluorescence emitting a first electromagnetic signal caused by two-photon excitation, the specimen contains non-linear materials to generate a second electromagnetic signal in the form of second harmonic wave pursuant to the irradiating step; recording the first and the second electromagnetic signals emitted from the specimen subjected to the irradiating step; co-localizing the recorded first and second electromagnetic signals to generate an image; analyzing the generated image to acquire one or more specimen data; and determining the presence of the diseased state by comparing the acquired specimen data with a corresponding reference data.

For some embodiments, the excitation wavelength of the light source ranges from 2.5 μm to 750 nm or at a frequency of 120 to 400 THz to realize the non-destructive approach to detect fibrous tissue deposited to the specimen.

In some embodiments, the one or more specimen data includes measurement on aggregated fiber percentage, measurement of total fiber number, measurement on total fiber area, measurement of total fiber width, measurement on total fiber length, measurement on total fiber cross-link and/or TPE/SHG ratio.

In a plurality of embodiments, the fibrous tissues related to diseased state can be determined through the disclosed method is myelofibrosis, nephrogenic systemic fibrosis, retroperitoneal fibrosis, mediastinal fibrosis, pulmonary fibrosis or endomyocardia fibrosis.

Another major aspect of the present disclosure refers to an image generating and analyzing system for quantifying presence of collagen fibers in a biological test specimen. Essentially, the disclosed system comprises a platform for deposition of the test specimen thereon; an electromagnetic light source coupling to the platform to direct a radiation at an excitation wavelength to an interested region of the test specimen, the radiation resulting in emission of a first and a second electromagnetic signals respectively caused by two-photon excitation (TPE) of excitable material and second harmonic generation (SHG) of optically non-linear materials in the collagen fibers in the test specimen; a first sensor and a second sensor being arranged to respectively real-time record the emitted first and second electromagnetic signals and convert the received first and second electromagnetic signals separately into a first digital signal and a second digital signal; an optical assembly for manipulating the radiation directed to the test specimen and the emitted first and second electromagnetic signals prior to having the first and second electromagnetic signals recorded by the first and second sensors; a microprocessor unit being configured to use the first and second electromagnetic signals in generating a co-localized image having a set of image properties and being indicative of spatial distribution of the collagen fibers in the interested region, quantify one or more scalar features and distribution features of the co-localized image to generate quantified result for each scalar and/or distribution feature, quantifying collagen deposition in the test specimen using the quantified results for the extracted scalar and distribution features; a visual display, in communication with the microprocessor unit, providing an interface comprising one or more modules for customizing the set of adjustable parameters, displaying the co-localized image, quantified results of the scalar features and distribution features, and quantified collagen deposition. Preferably, the distribution features of the co-localized image being quantified by the system are any one or combinations of intensity and/or distribution of the second electromagnetic signal, intensity and/or distribution of the first electromagnetic signal, gray level co-occurrence matrix (GLCM), thickness of collagen fiber, length of the collagen fibers, orientation of the collagen fibers, and straightness of the collagen fibers. Moreover, the scalar features being quantified in the disclosed system are any one or combinations of collagen proportion ratio, total average collagen, complexity of collagen fibers, and fragment average ratio of the collagen fibers.

In some embodiments, the excitation wavelength employed in the disclosed system is 700 to 850 nm. Preferably, the biological test specimen is trimmed to a thickness of 1 to 5 μm and free from any staining.

For more embodiments, the disclosed system has the microprocessor unit configured to compute an absolute total average collagen by way of subjecting the co-localized image to multiple iterations of enhancement that each iteration of enhancement corresponds to a quotient of enhancement including N-fold amplification of the default pixel intensity of the co-localized image, computing total average collagen for each iteration, generating a graft with the computed total average collagen plotting against the quotient of enhancement, identifying a inflexion point from the plotted graph, and referring the total average collagen on the graph corresponding to the identified inflexion point to derive the absolute total average collagen. Preferably, N is 0.1 to 100.

Also, in few embodiments, the microprocessor unit of the disclosed system is configured to identify collagen fibers associated to blood vessels of the test specimen generated in the co-localized image and exclude the identified collagen fibers associated to blood vessels in deriving the absolute total average collagen.

In more embodiments, the microprocessor unit of the disclosed system is configured to map the generated graph against a standard graph computed from a non-diseased specimen and derive a prognosis towards a diseased state associated to the test specimen based upon relative distance between the mapped generated graph and the standard graph, wherein the graphs are line graph.

For a number of embodiments, the microprocessor unit is configured to derive a prognosis towards a diseased state associated to the test specimen based upon a distance of the inflexion point of the generated graph in relation to a standard graph computed from a non-diseased specimen.

Further aspect of the present disclosure involves an image or micrograph, usable for assessing fibrosis state of a biological specimen or tissue sample associated with deposition of collagen fibers, generated by co-localizing a recorded first electromagnetic signal and a recorded second electromagnetic signal emitted from the specimen or sample pursuant to irradiation of the specimen or sample under an electromagnetic radiation of an excitation wavelength. The first electromagnetic signal is auto-fluorescence resulted from materials or compound, excited by two-photon excitation, contained within the specimen, and wherein the second electromagnetic signal is a second harmonic wave generated from non-linear materials pursuant to the irradiation. Preferably, the excitation wavelength of the light source can range from 2.5 μm to 750 nm or at a frequency of 120 to 400 THz to realize the non-destructive approach to detect fibrous tissue deposited to the specimen.

Another aspect of the present disclosure includes a method of evaluating a biological specimen or tissue sample for determination presence of a diseased state comprising the steps of illuminating the specimen using an electromagnetic radiation of an excitation wavelength that the specimen contains excitable materials or compound to result in auto-fluorescence emitting a first electromagnetic signal caused by two-photon excitation, the specimen contains non-linear materials to generate a second electromagnetic signal in the form of second harmonic wave pursuant to the illuminating step; recording the first and the second electromagnetic signals emitted from the specimen subjected to the illuminating step; co-localizing the recorded first and second electromagnetic signals to generate an image; analyzing the generated image to acquire one or more specimen data; and determining the presence of the diseased state by comparing the acquired specimen data with a corresponding reference data. Preferably, the one or more specimen data includes measurement on aggregated fiber percentage, measurement of total fiber number, measurement on total fiber area, measurement of total fiber width, measurement on total fiber length, measurement on total fiber cross-link and/or TPE/SHG ratio. The light source is preferably a laser beam or the like operable to induce two-photon excitation in at least part of the given tissue sample. Preferably, the reference data possesses measurements corresponding to the measurements of the specimen data to realize the comparison, which is preferably carried out by a fuzzy logic to attain an unbiased result, for diagnosing or determining the diseased state.

### BRIEF DESCRIPTION OF DRAWINGS

FIG. 1 illustrates and compares (a) one-photon excitation occurs through the absorption of a single photon and (b) two-photon excitation (2PE) occurs through the absorption of two lower-energy photons via short-lived intermediate states that fluorescence excitation is observed throughout the path of the laser beam in the single photon excitation occurs only within a 3-D localized spot for 2PE fluorescence (Image from Webb Lab, Cornell University, adapted by J. van Howe);

FIG. 2 illustrates simple working principle of second harmonic generation in generating a second harmonic wave;

FIG. 3 shows micrographs captured from (a) SPE and (b) SHG involving different contrast mechanisms that (c) a co-localized image/micrograph is generated utilizing complimentary information of (a) and (b) on cellular structure as well as function of unstained tissue sections;

FIG. 4 provides graphs generated by plotting consensus score against (a) fiber length, (b) fiber width, (c) fiber area and (d) aggregated fiber percentage computed from one embodiment of the disclosed method;

FIG. 5 shows and compares visual differences of micrographs showing (a) 0 consensus score, (b) 2+ consensus score, and (c) 3+ consensus score;

FIG. 6 illustrates fiber length density (FLD) calculation by manually counting the number of fibers that cross a fixed line distance, as in one arm of a fixed square, and the arrow points to a fiber being included in the FLD calculation;

FIG. 7 shows (a) a stained wedge kidney tissue, (b) a co-localized image generated using tissue in (a) by the present disclosed system before carrying out the staining and (c) processed co-localized image with the collagen-containing blood vessel excluded;

FIG. 8 presents line graphs (a) showing results of various samples subjected to enhancement according to a series of predetermined enhancement quotients and (b) showing identification of the absolute total average collagen or total collagen quantification through referring to the inflexion point;

FIG. 9 shows progression of fibrosis in kidney of mice treated with Cyclosporine A for (a) 0 days of treatment, (b) 7 days of treatment, (c) 14 days of treatment, (d) 21 days of treatment and (e) 28 days of treatment.

### DETAILED DESCRIPTION

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

The terms “light”, “photon”, “wave” or “electromagnetic signal” are used interchangeably throughout the specification referring to an electromagnetic wave of a particular spectrum carrying detectable or measurable power by any known method or apparatus in the field unless mentioned otherwise.

The terms “electromagnetic light source”, “light source” and “radiation source” are used interchangeably referring to an equipment used in the present disclosure to provide a radiation at a predetermined wavelength for excitation of a specimen leading to occurrence of TPE and SHG in the irradiated specimen and emitting electromagnetic signals usable for generating microscopy image with respect to cellular structure of the specimen.

The phrases relating to irradiating or radiation towards the biological specimen in the present specification generally refer to an action targeting to trigger a measurable spectroscopic or optical phenomenon in the biological specimen. Preferably, the measurable spectroscopic or optical phenomenon is, but not limited to, TPE and/or SHG.

With reference to one aspect, the present disclosure provides an image generating and analyzing system for quantifying presence of collagen fibers in a biological test specimen. The disclosed system is preferably nonlinear optical imaging system utilizing optical phenomenon such as TPE and SHG to collect useful cellular information and generate a co-localized image thereof. It essentially comprises a platform for deposition of the test specimen; an electromagnetic light source coupling to the platform to direct a radiation at an excitation wavelength to an interested region of the test specimen, the radiation resulting in emission of a first and a second electromagnetic signals respectively caused by two-photon excitation (TPE) of excitable material and second harmonic generation (SHG) of optically non-linear materials in the collagen fibers in the test specimen; a first sensor and a second sensor being arranged to respectively real-time record the emitted first and second electromagnetic signals and convert the received first and second electromagnetic signals separately into a first digital signal and a second digital signal; an optical assembly for manipulating the radiation directed to the test specimen and the emitted first and second electromagnetic signals prior to having the first and second electromagnetic signals recorded by the first and second sensors; a microprocessor unit being configured to use the first and second electromagnetic signals in generating a co-localized image having a set of image properties and being indicative of spatial distribution of the collagen fibers in the interested region, quantify one or more scalar features and distribution features of the co-localized image to generate quantified result for each scalar and/or distribution feature, quantifying collagen deposition in the test specimen using the computed quantified results for the extracted scalar and distribution features; and a visual display, in communication with the microprocessor unit, providing an interface comprising one or more modules for customizing the set of adjustable parameters, displaying the co-localized image, quantified results of the scalar features and distribution features, and quantified collagen deposition. Preferably, the biological test specimen is trimmed to a thickness of 1 to 5 μm and free from any staining.

According to several preferred embodiments, the platform can be a housing, casing or the like in which at least some of the components including both electronic and optical elements are stored, connected and/or mounted to facilitate irradiation of the test specimen and subsequent registration of the optical or electromagnetic signals emitted from the irradiated materials. More preferably, the platform may possess a reception port carrying an openable lid for the user to place the test sample into the port and subject the placed sample to irradiation. The openable lid is preferably made of material which is substantially impermeable to the radiation. A control panel with physical button and/or tactile input means can be fabricated on the platform in some embodiments permitting adjustment to be made with respect to the radiation and signal capturing parameters, on-site storing of the captured signals and/or generation of the co-localized image without the need of using a remotely connected visual display.

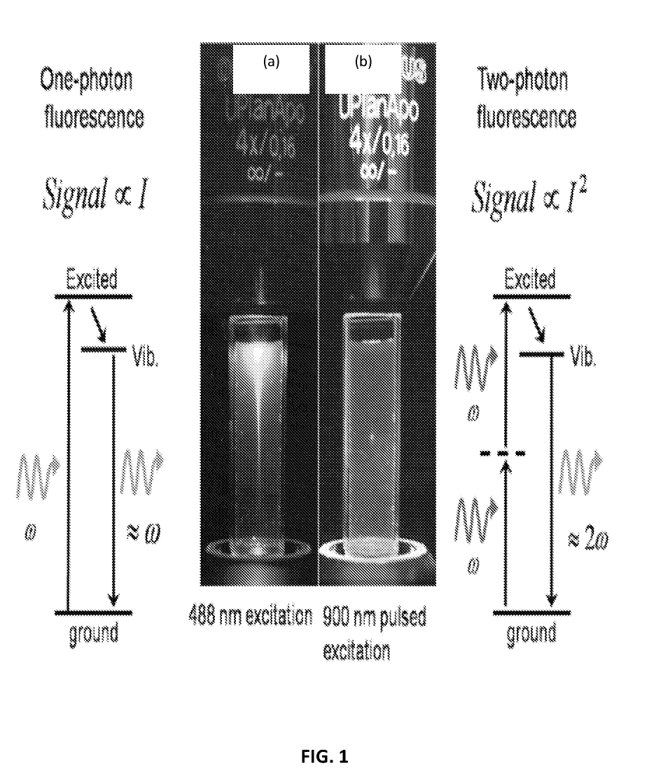

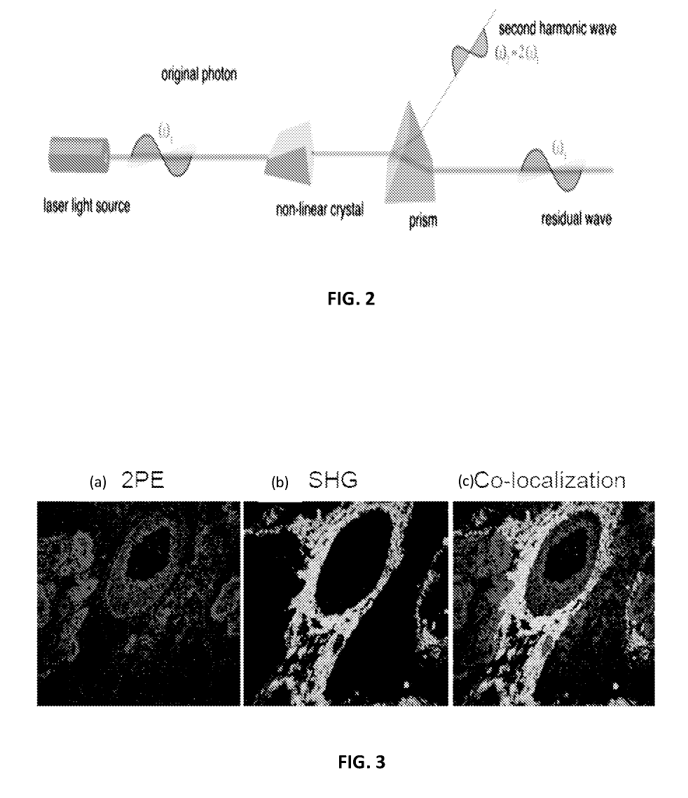

In some preferred embodiments, the light source generates an excitation light or radiation towards the test specimen at a predetermined excitation wavelength. The light or radiation source to be used for irradiating the specimen or sample is preferably generated using a focused, pulsed laser beam to excite endogenous fluorophores. In TPE, two photons of lower energy are simultaneously absorbed by a molecule in the specimen. If the excited molecule is fluorescent, it will emit a photon just as one would expect from the absorption of a single photon of greater energy. However, unlike one-photon confocal microscopy, TPE can use low energy photons to penetrate deeper into tissue with no absorption in out-of-focus areas as illustrated in the comparison made in FIG. 1. Excitation occurs only at the focal point of the laser where photon density is greatest resulting in less risk of tissue damage. In addition, TPE allows for stain-free quantification of unstained tissue structures as a result of intrinsic auto-fluorescence emissions. More preferably, the light source in the disclosed system can be a laser light source or pulsed-laser light source tunable to provide a range of radiation having desired wavelength and power. The radiation or excitation wavelength preferably causes relatively low light scattering in the irradiated test sample, such that the micrographs and/or co-localized image acquired has better resolution and lower background noise. Preferably, the excitation wavelength employed in the disclosed system ranges from 700 to 850 nm to attain minimal interference caused by unwanted light scattering in the test specimen. As stated in the foregoing, the radiation or light at the desired wavelength being directed to the specimen shall give rise to emission of the first and the second electromagnetic signals respectively caused by TPE of excitable or auto-fluorescent material and SHG of optically non-linear materials from the collagen fibers in the test specimen. More preferably, the TPE and SHG signals emitted from the test sample may be separately recorded from two or more radiations conducted at different excitation wavelength for a number of preferred embodiments. TPE or SHG related signals are selectively registered according to the excitation wavelength used to ensure more data can be collected through the optical phenomenon triggered. Still, in more embodiments, both TPE and SHG derived signals are in fact recorded in each radiation of the specific excitation wavelength, but the disclosed system samples the SHG- and TPE-related signals respectively from signals recorded in two or more radiations performed at different excitation wavelengths and subsequently combines the sampled signals to compose the co-localized images. Furthermore, the disclosed system may be configured to subject the test specimen for multiple irradiations with each irradiation performed at different excitation powers that signals correspondingly emitted from test specimen irradiated at a given incident excitation power are captured to create image of that particular excitation power. User of the disclosed system can then select the best generated image or micrograph for subsequent analysis. The optical assembly may comprise an internal power meter to gauge the power output of the laser before channeling the light or radiation to the test specimen.

It is important to note that the optical assembly in the disclosed system is a multi-components construct. Some components of the optical assembly are disposed in the light path of the radiation propagating towards the test specimen. The optical assembly intercepts and manipulates the light properties of the radiation. Also, some other components of the optical assembly process the electromagnetic signals emitted from irradiated specimen before allowing the electromagnetic signals to be registered by the sensors. For instance, an XY scanning mirror is disposed at the outlet of the electromagnetic radiation source in some embodiments to steer the laser beam originated thereof to the test specimen. Further embodiments of the disclosed system carry an optical modulator, as part of the optical assembly, being positioned between the radiation or light source and the XY scanning mirror. The optical modulator controls and regulates the power of the radiation finally guided to the interested region of the test specimen. Unregulated high power of the excitation light towards the test specimen can lead to denaturing of the specimen, particularly at the region exposed to the excitation light or electromagnetic radiation. More preferably, a set of objective lens may be used as well in few embodiments of the disclosed system to focus the light or radiation to the interested region of the test specimen for triggering the TPE and/or SHG in the specimen. In a number of embodiments, the TPE- and SHG-derived electromagnetic signals are emitted in substantially different or even opposite directions that corresponding arrangements have been made in the optical assembly to process, filter and/or manipulate the emitted signal before having the emitted signals registered by the designated sensors. For SHG-derived second electromagnetic signals propagating in a direction parallel to the radiation, the optical assembly of the disclosed system includes an optical condenser oriented to collect the second electromagnetic signals. The optical condenser is fabricated to have a numerical aperture of 0.4-0.9 for optimal signal collection efficiency and acceptable field of depth as well in the tissue image generated. An optical filter is located immediately downstream of the optical condenser permitting only the second electromagnetic signals to pass through while prohibiting potentially the first electromagnetic signal and stray light of the radiation source from reaching the second sensor. On the other hand, the TPE-related or the first electromagnetic signals are emitted in a direction substantially opposing to the forwarding radiation. Likewise, the disclosed system uses one or more optical filters to isolate the first electromagnetic signal from the other interference before letting the first electromagnetic signals to be registered by the first sensor. One skilled artisan in the field shall appreciate the fact that it is possible to use mirror, prism or other optical elements to change light path of the emitted signals towards the sensors; the forward or backward propagation of the emitted signals described in this specification merely facilitate understanding about general embodiments of the disclosed system without limiting scope of the present disclosure.

The first or second sensor setting out the present disclosure can refer to one or more sensors dedicated to detect and capture the desire electromagnetic signals. For example, the first electromagnetic signals are detected by the first sensor in the form of charge coupled device (CCD) or photomultiplier tube (PMT) in several preferred embodiments. Furthermore, multiple CCD and/or PMT may be employed in the disclosed system that each installed CCD or PMT is fashioned to register signals output at different excitation wavelength. Similar setup is applicable in the second sensor for detecting and capturing the second electromagnetic signals. The first and second sensors of the present disclosure are also responsible for converting the analogue wave or light signals emitted into digital signals preferably through an analogue to digital convertor coupled to the sensor.

As setting forth in the above description, the microprocessor unit in the disclosed system is configured to use the first and second electromagnetic signals in generating a co-localized image having a set of image properties and being indicative of spatial distribution of the collagen fibers in the interested region. Particularly, the test specimen contains cellular materials or endogenous fluorophores, which are capable of absorbing two low energy exciting photos provided by the excitation radiation and discharging energy from the absorbed exciting photos in the form of a single discharged photon. The discharged photons from the TPE correspond to the first electromagnetic signals. As such, the first electromagnetic signals collected are suggestive of cellular structure distribution in the interested region excitable by the radiation beam and usable for producing a first micrograph illustrative of the cellular structure become fluorescent in connection to the irradiation. In addition, the two exciting photons entering the specimen can be interacted or combined by optically non-linear material such as collagen resulting in the second harmonic generation phenomenon give rise to the second harmonic wave, which corresponds to the second electromagnetic signal described throughout this specification. A prerequisite for SHG is that the specimen must exhibit a high degree of nonlinear molecular organization in order to generate appreciable SHG signals. Therefore, the SHG-related spectroscopic phenomenon is resorted in the disclosed system to acquire useful information with respect to diseases associated with fibrosis such as kidney fibrosis, lung fibrosis, bone marrow fibrosis and/or liver fibrosis. Like the first electromagnetic signals, the second electromagnetic signals collected in the disclosed system are applicable for composing a second micrograph indicative of degree or progression of fibrosis in the irradiated test specimen. Owing to the fact that TPE and SHG involve different contrast mechanisms, the microprocessor unit can therefore use the first and second electromagnetic signals in tandem to provide complimentary information regarding cellular structure and collagen fibers. In more specific, the microprocessor unit generates the co-localized image by way of mapping the first micrograph to the second micrograph or vice versa. In a plenty of embodiments, the microprocessor unit directly generates the co-localized image using the first and second digital signals. For several preferred embodiments, the generated co-localized image has a spatial resolution of 0.05 μm to 1 μm, or more preferably 0.1 μm to 0.5 μm.

Pursuant to other preferred embodiments, the microprocessor unit in the disclosed system further extracts and analyzes distribution and/or scalar features of the co-localized image created thus quantifying the collagen fibers deposited and/or correlating a diseased state to the test specimen being imaged. It is important to note the disclosed system may employ multiple microprocessor units in the image generation and analysis. For instance, the platform may house a relatively low computing power microprocessor unit to collect and process the first and second digital signals, while then digital signals are then transferred or transmitted in the form of digital data to a remotely connected work station or server carrying microprocessor unit of much higher computing power to perform the scalar and/or distribution features analysis. The remotely connected work station or server is part of the disclosed system in such embodiments. The microprocessor unit preferably couples to a communication module capable of running any known network protocol to effectuate the data transmission.

Also, the microprocessor units preferably run the features extraction and/or analysis based upon one or more computer program executable by the microprocessor units. One or more algorithms developed by inventors of the present disclosure are integrated into the mentioned computer program to realize the analysis on the extracted features and, optionally, conclude on progression of an identified diseased state regarding to the test specimen. In a number of embodiments, the distribution features of the co-localized image being extracted and analyzed are any one or combinations of intensity and/or distribution of the second electromagnetic signal, intensity and/or distribution of the first electromagnetic signal, gray level co-occurrence matrix (GLCM), thickness of collagen fiber, length of the collagen fibers, orientation of the collagen fibers, and straightness of the collagen fibers. More specifically, the distribution or intensity of the second electromagnetic signal corresponds to brightness of collagen fibers in the co-localized image generated and is relatively indicative of the amount of collagen fibers deposited to the interested region in the specimen. The external structure, shape or pattern of the other cellular structures located in the interested region can be revealed by intensity and/or distribution of the first electromagnetic signal, which is complement to the second electromagnetic signal in the co-localized image. Further, the GLCM is employed in the disclosed system for texture classification of the identified collagen fibers that correlation and dissimilarity of the spots sampled from the co-localized image are preferably computed and plotted in the feature space to determine the class. The disclosed system may possess a classifier module or machine trainable to conduct the classification. Signal intensity-based distribution features aside, the disclosed system also extracts and computes quantified result using structure based distribution features such as thickness, length, orientation, straightness of the identified collagen fibers.

More preferably, the microprocessor unit further computes quantified results from at least one of mean, variance, skewness, kurtosis, energy and entropy for each of the aforesaid distribution features. The mean value computed is representative of average value of the feature distributed in the co-localized image. The variance value is square of the distribution standard deviation for quantifying dispersion of the feature distribution in the co-localized image. The skewness enables the disclosed system to measure symmetry or asymmetry of the distribution and the kurtosis provides information about the tail of the distribution. Value quantified or computed on energy allows the disclosed system to ascertain intensity variation in the co-localized image that extremely low energy can associate to one or more diseased state. Further, quantified result of the entropy is representative of random distribution of the collagen fibers in the acquired image that high entropy can be disease-related. By computing statistical value of at least one of mean, variance, skewness, kurtosis, energy and entropy, the maximal number of quantified results for distribution features can be 42 in total for several embodiments of the disclosed system.

In addition, the scalar features extracted from and analyzed about the co-localized image are any one or combinations of collagen proportion ratio (CPR), total average collagen (TAC), complexity of collagen fibers, and fragment average ratio (FAR) of the collagen fibers. More specifically, the CPR refers to a ratio of the area occupied by the collagen fibers or collagen over the area of the interested region and applicable for the disclosed system to evaluate the area, more precisely percentage of area, in the specimen being occupied by the deposited collagen fibers. CPR can be generally summarized in the equation (1) given below:

$$CPR = \frac{{A}_{cn}}{{A}_{Roi}} , \qquad Equation ( 1 )$$

where A_{cn} is area occupied by collagen and A_{Roi} is area of the interested region.

In meantime, TAC is a ratio of the sum of collagen intensity over each pixel in the area occupied by collagen or collagen fibers. The disclosed system preferably measures brightness or intensity of the brightness with respect to collagen in the co-localized image. The TAC can be summarized as

$$TAC = \frac{{Σ}_{pix} {I}_{cn}}{{A}_{cn}} , \qquad Equation ( 2 )$$

where I_{cn} is intensity of a given pixel in the area covered by collagen and A_{cn} is area occupied by collagen.

Further, the complexity computed by the disclosed system refers to a ratio of the number of branches node over the total skeleton area that skeleton is defined as a thinning binary process reducing the collagen fibers to a line connected by only one pixel. This measurement quantifies morphology of the collagen fibers in terms of branching and/or straightness. Computed value of the complexity becomes closer to zero when there are less branches or the collagen fibers are not connected one another in the co-localized image. Complexity corresponds to Equation (3) set out in the following:

$$Complexity = \frac{{N}_{bp}}{{N}_{skel}} , \qquad Equation ( 3 )$$

where N_{bp} is number of braches node and Nₛₖₑₗ is number of skeleton area.

In several embodiments, the disclosed system computes the FAR by dividing the number of connected fibers (bundle of fibers) over the area of the interested region. The computed value of FAR indicates connectivity of the collagen fibers in relation to the whole area of the interested region. Preferably, FAR can be generally represented by Equation (4) as stated below:

$$FAR = \frac{{N}_{segments}}{{A}_{Roi}} , \qquad Equation ( 3 )$$

where N_{segments} is the number of connected collagen fibers and A_{Roi} is area of the interested region.

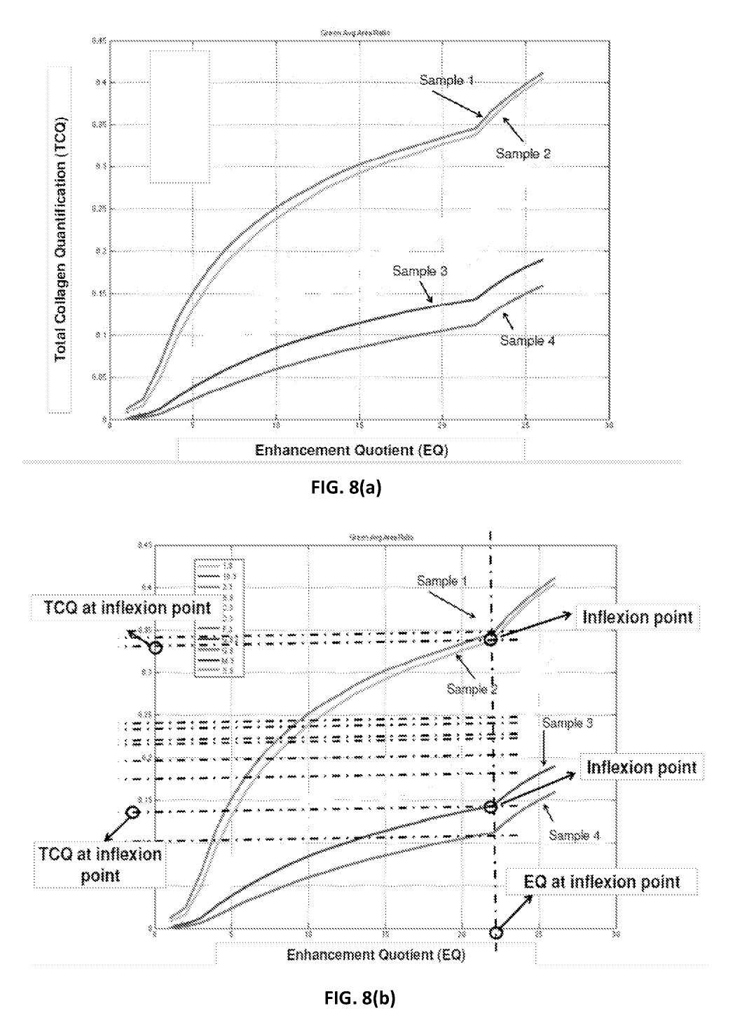

As aforementioned, the TAC value correlates to both number of pixel and intensity of the pixel associated to the collagen fibers in the interested region or the second electromagnetic signals registered. The disclosed system preferably adopts default parameters in composing the co-localized image such that the image is indicative and illustrative of the collagen fibers spatial arrangement in the interested region as precise as possible. The default parameters implemented ensure significant background noises are filtered off in the composed image. Still, there are scenario in which the intensity of one or more particular pixels in the composed co-localized image are too low and being disregarded as genuine signals registered with regard to the second electromagnetic signals. These overlooked low intensity pixels can be in significant numbers especially at early stage of a diseased state manifested in the test specimen that excluding these pixels in the analysis will inevitably lead to false negative result depriving the patient from the opportunity of timely therapy. Anyhow, it is possible to further manipulate image properties of the composed co-localized image to bring forth the weak signal or low intensity pixel in computing TAC, more precisely the absolute TAC, substantially free from any adverse impact from noise or other interferences. To derive the absolute TAC, the microprocessor subjects the co-localized image to multiple iterations of enhancement that each iteration of enhancement corresponds to a quotient of enhancement including N-fold amplification of the default pixel intensity of the co-localized image, computes the total average collagen for each iteration, generates a graft with the computed total average collagen plotting against the quotient of enhancement, identifies a inflexion point from the plotted graph, and refers the total average collagen on the graph corresponding to the identified inflexion point to derive the absolute total average collagen. Preferably, N is 0.1 to 100 relying upon tissue types of the specimen and also the embodiment of the disclosed system being implemented. More specifically, the quotient of enhancement or enhancement quotient (EQ) involves changes made to a set of image properties towards the composed image. Each attempt of EQ involves enhancement of each desired image property according to a predetermined multiplicative factor or algorithm until the inflexion point of the produced graph can be determined, as illustrated in FIGS. 8a and 8b. It was found by inventors of the present disclosure that progressive enhancement such as amplification of pixel intensity in EQ substantially exhibits a natural log relationship in the produced graph until it exceeds a particular juncture, preferably the inflexion point, that background noises and/or interferences starting to flare up and play significant part in the overall intensity of the composed image. Therefore, the inflexion point in the plotted graph becomes an ideal reference point for the disclosed system to best determine absolute TAC in the image based on optimal emitted second electromagnetic signals with minimal amplification on noises and interferences.

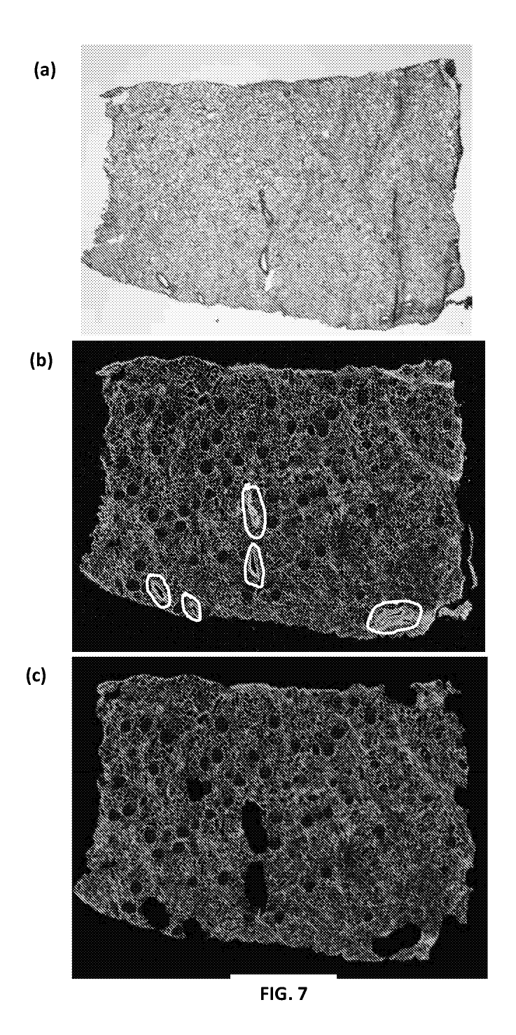

Taking into consideration that collagen fibers are intrinsic components in blood vessel, TAC or absolute TAC computed inclusive of collagen fibers in blood vessel may yield false positive result or imprecise prognostic on progression of a fibrosis-related disease. Furthermore, profuseness or distribution of blood vessel can be significantly varied even in a single organ or tissue type. It is almost impossible to simply factor out collagen in blood vessel using a predetermined baseline figure. To resolve false result possibly caused by intrinsic collagen in blood vessel, the disclosed system is equipped with the capability to identify and subsequently exclude collagen content of the blood vessel appeared in the composed co-localized image. In more specific, the microprocessor unit is configured to identify collagen fibers associated to blood vessels of the test specimen generated in the co-localized image and exclude the identified collagen fibers associated to blood vessels in deriving the absolute total average collagen. Again, inventors of the present disclosure discovered that the blood vessel structure is registered under a light spectrum or contrast different from the collagen fibers by the second sensor in connection to the SHG. For example, in some preferred embodiments, the collagen fibers are highlighted in green and cellular structures made illustrative by TPE are in red, while the blood vessel is illuminated as yellow under default setting in the composed co-localized image. With the discernible differences in the registered light spectrum, the disclosed system regards the collagen fibers overlapping with the identified blood vessel in the co-localized image as the intrinsic collagen fiber. Preferably, the identified blood vessels and the collagen fibers encompassed within the blood vessel are removed from the co-localized image by the disclosed system before subjecting the image to features extraction and analysis as shown in FIG. 7(a)-(c).

Besides providing reference point relating to absolute TAC, the computed graph for determining the inflexion point is applicable as well in deriving a preliminary diagnostic or prognostic result with respect to progression of a diseased state. More specifically, position of the graph plotted for the test specimen in relation to a standard graph produced from a non-diseased specimen can provide empirical data to draw a prognostic conclusion, optionally in the presence of fuzzy logic embedded to the microprocessor unit. Preferably, the microprocessor unit of the disclosed system is configured to map the generated graph against the standard graph computed from a non-diseased specimen and derive a prognosis towards a diseased state associated to the test specimen based upon relative distance between the mapped generated graph and the standard graph. More preferably, the generated and standard graphs are line graph.

Likewise, the disclosed system can draw a prognostic conclusion using relative position of the inflexion point in the graph computed for test specimen through a comparison toward an inflexion point in a standard graph generated from a non-diseased sample. Preferably, the microprocessor unit is configured to derive a prognosis towards a diseased state associated to the test specimen based upon a distance of the inflexion point of the generated graph in relation to a standard inflexion point in a standard graph computed from a non-diseased specimen. The relative distance of the inflexion points provide empirical information about the diseased state. Particularly, the more the inflexion point of the test specimen deviated or distanced from the standard inflexion point the more critical the condition is.

Referring to another aspect of the present disclosure, a method of image generating and analyzing is described hereinafter. More specifically, the disclosed method is directed for quantifying collagen fibers in a biological test specimen to preferably attain a prognostic or diagnostic result using the image generated and analyzed. The disclosed method generally comprises the steps of irradiating an interested region of the test specimen using an electromagnetic light source through an optical assembly at an excitation wavelength, the irradiated test specimen having excitable materials and optically non-linear materials in the collagen fibers respectively leading to concurrent emission of a first electromagnetic signal caused by TPE and a second electromagnetic signal as a result of SHG; manipulating the first and second electromagnetic signals emitted from the test specimen through the optical assembly; recording the manipulated first and the second electromagnetic signals through one or more sensors; using the concurrently recorded first and second electromagnetic signals to generate a co-localized image having a set of image properties and being indicative of spatial distribution of the collagen fibers in the interested region; quantifying one or more scalar features and distribution features of the co-localized image to generate quantified result for each scalar and/or distribution feature; and quantifying deposition of collagen fibers in the test specimen using the calculated quantified results for the extracted scalar and distribution features.

In several embodiments, the disclosed method employs a laser light source or pulsed-laser light source tunable to provide radiation having desired wavelength to excite endogenous fluorophores in the specimen. By using the radiation light source, the disclosed method directs at least two photons of lower energy simultaneously into and be absorbed by a molecule in the specimen to create TPE in the irradiated specimen. TPE in the disclosed method can use low energy photons to penetrate deeper into tissue with no absorption in out-of-focus areas. Excitation occurs only at the focal point, which is preferably adjustable through an optical assembly having an objective lens, of the laser or light where photon density is greatest, resulting in less risk of tissue damage. The radiation is preferably at an excitation wavelength causing relatively low light scattering in the irradiated test sample, such that the micrographs and/or co-localized image acquired has better resolution and lower background noise. Preferably, the excitation wavelength employed in the disclosed method ranges from 700 to 850 nm to attain minimal interference caused by the unwanted light scattered in the test specimen.

As stated in the foregoing, the radiation or light at the desired wavelength being directed to the specimen shall give rise to emission of the first and the second electromagnetic signals respectively caused by TPE of excitable or auto-fluorescent material and SHG of optically non-linear materials such as collagen fibers in the test specimen. More preferably, the TPE and SHG signals emitted from the test sample may be separately recorded from two or more radiations conducted at different excitation wavelength for a number of preferred embodiments. TPE or SHG related signals are selectively registered according to the excitation wavelength used to ensure more data can be collected through the optical phenomenon triggered. Still, in more embodiments, both TPE and SHG derived signals are recorded in each radiation of the specific excitation wavelength, but the disclosed system samples the SHG- and TPE-related signals respectively from data recorded in two or more radiations of different excitation wavelengths then combines the sampled data to compose the co-localized images. Still, the disclosed method may subject the test specimen for multiple irradiations with each irradiation performed at different excitation power and corresponding signals emitted from test specimen irradiated at a given incident excitation power are captured to create image of that particular excitation power. The disclosed method allows selecting the best generated image or micrograph thereafter for analysis.

To manipulate the emitted first and second electromagnetic signals, an optical assembly having a plurality of optical elements is used in the disclosed method. For example, the optical assembly used in the disclosed method includes an optical condenser oriented to collect the second electromagnetic signals emitted from the irradiated test specimen. The optical condenser is fabricated to have a numerical aperture of 0.4-0.9. The disclosed method also preferably has an optical filter placed immediately downstream of the optical condenser permitting only the second electromagnetic signals to pass through while prohibiting potentially the first electromagnetic signal and stray light of the radiation source from reaching the second sensor. As mentioned above, the TPE-related or the first electromagnetic signal is emitted in a direction substantially opposing to the second electromagnetic signal that one or more optical filters are used in the disclosed method to isolate the first electromagnetic signal from the other interference before letting the first electromagnetic signals to be registered by the sensor. The disclosed method may use two sensors, namely a first and a second sensor, to separately capture the first and second electromagnetic signals. Additionally, the optical assembly in the present method also engages in manipulating the radiation or light output from the radiation light source before reaching the specimen. For instance, an XY scanning mirror may be disposed at the outlet of the electromagnetic radiation source to steer the laser beam originated thereof to the test specimen in some embodiments of the disclosed method. Also, an optical modulator, as part of the optical assembly, can be positioned between the radiation or light source and the XY scanning mirror to control the power of the radiation finally directed towards the interested region of the test specimen to prevent denaturation of the specimen in connection to high power heating. More importantly, a set of objective lens incorporated into the optical assembly too for focusing the light or radiation to the interested region of the test specimen succeeded by the TPE and/or SHG triggered in the specimen.

Preferably, the disclosed method subsequently generates the co-localized image using the first and second electromagnetic signals captured. As explained in the foregoing, some cellular materials or endogenous fluorophores in the specimen absorbs two low energy exciting photos provided by the excitation radiation and discharges one photon of relatively higher energy than the exciting photons. The discharged photons from TPE correspond to the first electromagnetic signals. The first electromagnetic signals can be used by the disclosed method to compose a first micrograph being illustrative of cellular structures become fluorescent under TPE. In addition to TPE, the radiation initiates SHG in optically non-linear materials such as collagen the specimen giving rise to the second harmonic wave, which corresponds to the second electromagnetic signal described throughout this specification. Like the first electromagnetic signals, the second electromagnetic signals collected in the disclosed method are applicable for composing a second micrograph indicative of degree or progression of fibrosis in the irradiated test specimen. The first micrograph of TPE and the second micrograph of SHG can be mapped onto each another to form the co-localized image usable for assessment of collagen deposition and/or fibrosis-related disease. In a plenty of embodiments, the co-localized image composed in the present method has a spatial resolution of 0.05 μm to 1 μm, or more preferably 0.1 μm to 0.5 μm.

In order to quantify collagen fibers in the specimen and possible making a correlation towards a suspected diseased state, the disclosed method extracts and analyzes distribution and/or scalar features of the co-localized image. According to the preferred embodiments, the distribution features of the co-localized image being extracted and analyzed in the disclosed method can be any one or combinations of intensity and/or distribution of the second electromagnetic signal, intensity and/or distribution of the first electromagnetic signal, gray level co-occurrence matrix (GLCM), thickness of collagen fiber, length of the collagen fibers, orientation of the collagen fibers, and straightness of the collagen fibers. Apart from using signal intensity-based distribution features, the disclosed method also extracts and calculates quantified result using structure based distribution features such as thickness, length, orientation, straightness of the identified collagen fibers. More preferably, the disclosed method also calculates or acquires results from at least one of mean, variance, skewness, kurtosis, energy and entropy for each of the aforesaid distribution features. As set forth earlier, mean value represents average value of the feature distributed in the co-localized image, variance value characterizes dispersion of the feature distribution, skewness enables measurement on symmetry or asymmetry of the distribution, kurtosis provides information about the tail of the distribution, energy provides insight about intensity variation in the co-localized image and entropy is representative of random distribution of the collagen fibers in the acquired image. Moreover, the scalar features extracted from and analyzed in the disclosed method can be one or combinations of collagen proportion ratio (CPR), total average collagen (TAC), complexity of collagen fibers, and fragment average ratio (FAR) of the collagen fibers. Results or numerical figures of the mentioned scalar features with regard to the co-localized image analyzed can be derived from Equation 1-4 as stated in earlier descriptions.

In some embodiments, the disclosed method further derives the absolute TAC. Particularly, the disclosed method subjects the co-localized image to multiple iterations of enhancement that each iteration of enhancement corresponds to a quotient of enhancement including N-fold amplification of the default pixel intensity of the co-localized image, calculates the total average collagen for each iteration, generates a graft with the calculated total average collagen plotting against the quotient of enhancement, identifies a inflexion point from the plotted graph, and refers the total average collagen on the graph corresponding to the identified inflexion point to derive the absolute total average collagen. Preferably, N is 0.1 to 100. More specifically, the quotient of enhancement or enhancement quotient (EQ) involves changes made to a set of image properties towards the composed image. The inflexion point in the plotted graph is a reference point employed in the present method to best determine absolute TAC in the co-localized image based on optimal emitted second electromagnetic signals with minimal amplification on the noises and interferences. Also, the disclosed method set out to resolve false result possibly caused by intrinsic collagen in blood vessel as described earlier in a number of embodiments. More specifically, the disclosed method comprises the step of identifying collagen fibers associated to blood vessels of the test specimen generated in the co-localized image and excluding the identified collagen fibers associated to blood vessels in deriving the absolute total average collagen. The disclosed method relies on discernible differences in the registered light spectrums between the blood vessel and the collagen fibers to identify the blood vessel then regards the collagen fibers overlapping with identified blood vessel in the co-localized image as the intrinsic collagen fiber. Preferably, the identified blood vessels and the collagen fibers encompassed within the blood vessel are removed by the disclosed method from the co-localized image before subjecting the image to features extraction and analysis.

Accordingly, the plotted graph can be employed by the disclosed method as well in deriving a preliminary diagnostic or prognostic result with respect to progression of a diseased state. More specifically, position of the graph plotted for the test specimen in relation to a standard graph produced from a non-diseased specimen can provide empirical data adequate for drawing a prognostic conclusion. More preferably, the disclosed method maps the generated graph against the standard graph calculated from a non-diseased specimen and derives a prognosis towards a diseased state associated to the test specimen based upon relative distance between the generated graph and the standard graph. Alternatively, the disclosed method can draw a prognostic conclusion using relative position of the inflexion point in the graph generated for test specimen compared to an inflexion point in a standard graph generated from a non-diseased sample. Preferably, the disclosed method derives a prognosis towards a diseased state associated to the test specimen based upon a distance of the inflexion point of the generated graph in relation to a standard inflexion point in a standard graph calculated from a non-diseased specimen. The relative distance of the inflexion points provide empirical information about the diseased state.

The following example is intended to further illustrate the invention, without any intent for the invention to be limited to the specific embodiments described therein.

### Example 1

Bone marrow fibrosis is routinely assessed in the diagnostic work-up and prognostic evaluation of patients with known or suspected myeloproliferative neoplasm (MPN). Conventional evaluation of bone marrow fibrosis in cases of MPN is performed on reticulin- and trichrome-stained slides and is semi-quantitative and subjective. MPN grading systems, including the recently revised European consensus (EC) scoring system that uses a 0-3 scale, have been shown to have numerous limitations, including interobserver variability due to the subjective nature of scoring stained slides. The present disclosure exploit the applicability of two-photon excitation/second harmonic generation laser scanning microscopy (2PE/SHG) for quantification of fibrosis in unstained bone marrow core biopsy samples and compared its performance to the EC scoring system and a stereology-based quantitative method.

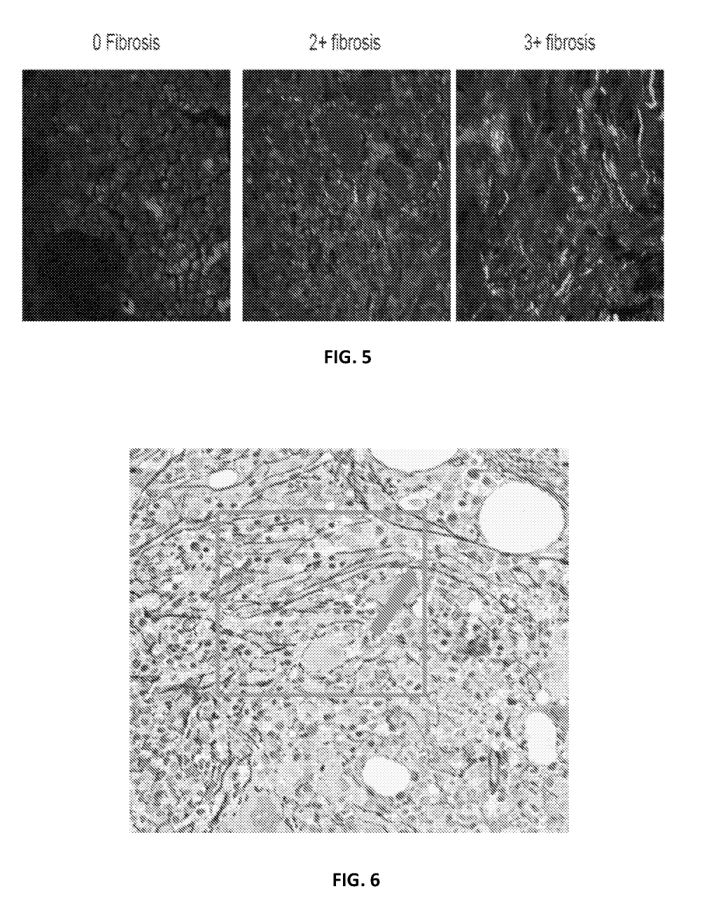

Bone marrow core biopsy samples submitted with an indication of MPN were selected or the study. An experienced hematopathologist reviewed the reticulin-stained slides and confirmed the EC score for 8 samples selected for study inclusion. The European consensus scores of the study samples included 0, 2+ and 3+ fibrosis as shown in FIG. 5.

Unstained 4 μm sections tissue sections underwent 2PE/SHG using the Genesis® 200 (Histoindex Pte, Ltd., Singapore). The Genesis® 200 utilizes an femtosecond erbium fiber laser with an excitation wavelength of 780 nm. A 20× objective was used to acquire multi-tiled images (600 μm2 total area) from each sample, resulting in a spatial resolution of approximately 0.2 μm. Image analysis was performed using proprietary software developed by Histoindex. Analysis parameters assessed included 2PE/SHG ratio, aggregated fiber percentage, total number, area, width, and length of fibers, and number of fiber cross-links.

Following 2PE/SHG imaging, the tissue sections were stained with reticulin and scanned using the Aperio ScanScope (Leica Biosystems, Buffalo Grove, Ill.). Fiber length density (FLD) was calculated using an approach adapted from stereology. To calculate FLD, the number of reticulin-stained fibers that crossed over a fixed line distance of 239.6 μm was manually counted (FIG. 6).

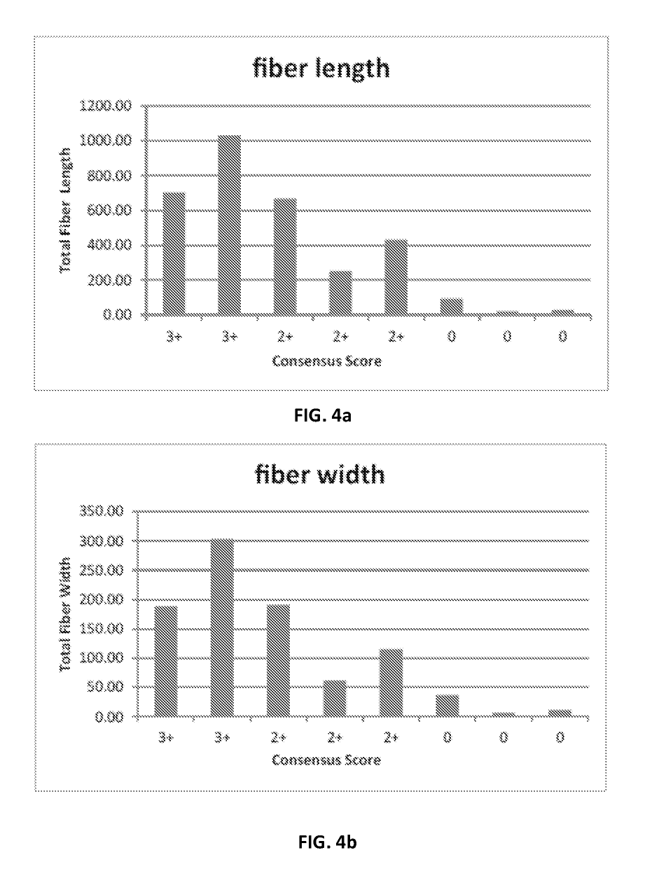

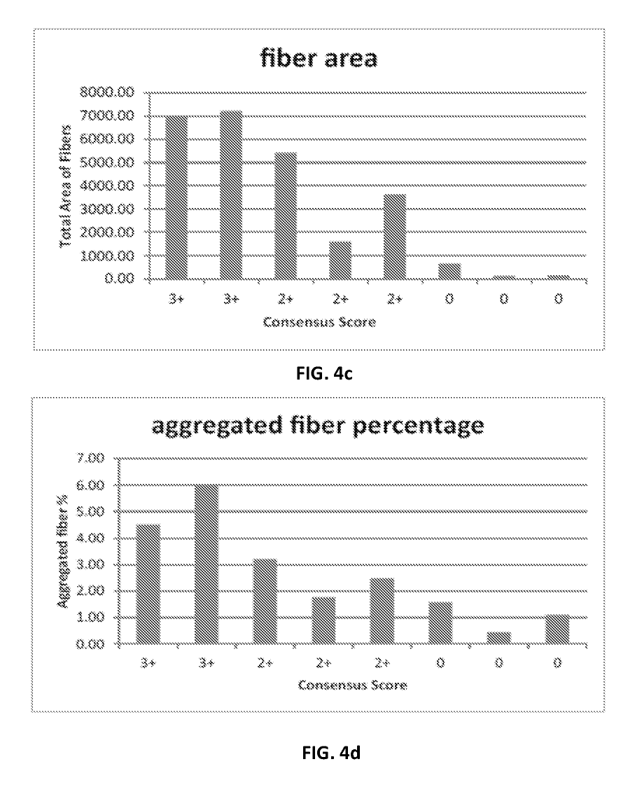

2PE/SHG imaging segregated unstained bone marrow biopsy core samples with fibrosis from those without fibrosis. Graphs, as presented in FIG. 4a-4d, were plotted for 2PE/SHG analysis and EC scores. Binomial logistic regression showed a high degree of correlation between 2PE/SHG analysis and EC score for the majority of the parameters evaluated (Table 1). Total area of fibers (p=0.004) and total length of fibers (p=0.008) demonstrated the most significant degree of correlation. Interestingly, FLD using a stereology-based approach, also showed a significant correlation (p=0.001) with the EC score. Cross-linking of fibers showed a trend but did not reach statistical significance (p=0.062).

TABLE 1
Correlation between 2PE/SHG image analysis and European
consensus score

| 2PE/SHG parameter | p-value |
|---|---|
| 2PE/SHG ratio | 0.054 |
| aggregated fiber percentage | 0.015 |
| total number of fibers | 0.023 |
| total area of fibers | 0.004 |
| total width of fibers | 0.016 |
| total length of fibers | 0.008 |
| number of fiber cross-links | 0.062 |

2PE/SHG image analysis is a promising novel technique to be applied in the quantification of bone marrow fibrosis with performance equivalent to a stereology-based approach. This method produces images with the resolution of standard histology and eliminates the subjective scoring associated with grading trichrome- and reticulin-stained slides. Further studies with large sample size are needed to validate the utility of this method, however; several parameters can now be evaluated, as well as compared, to the biology of MPNs.

### Example 2

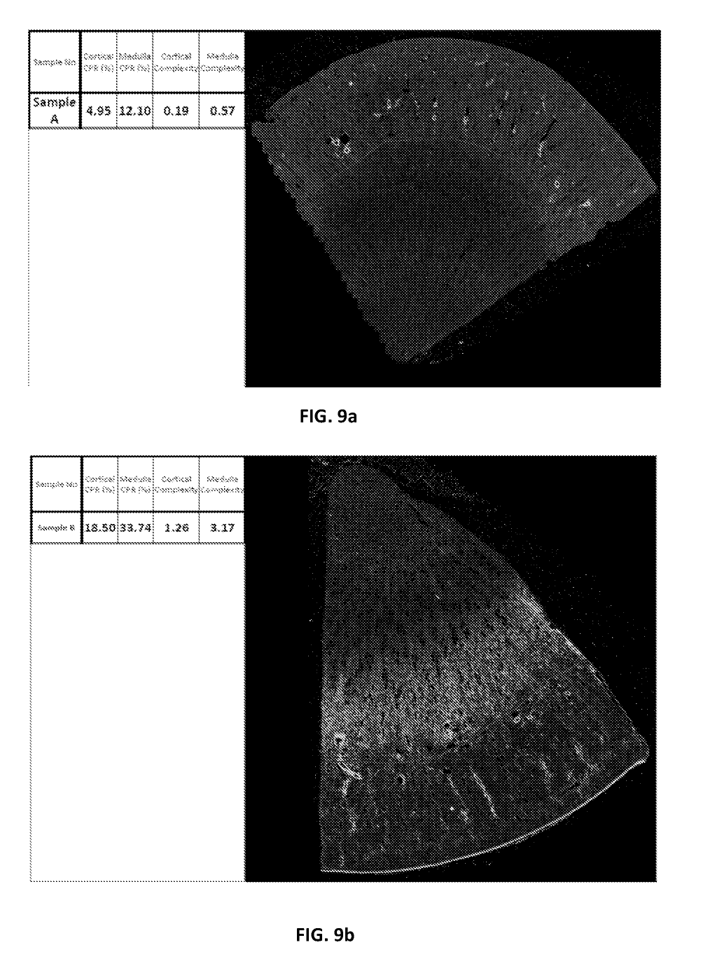

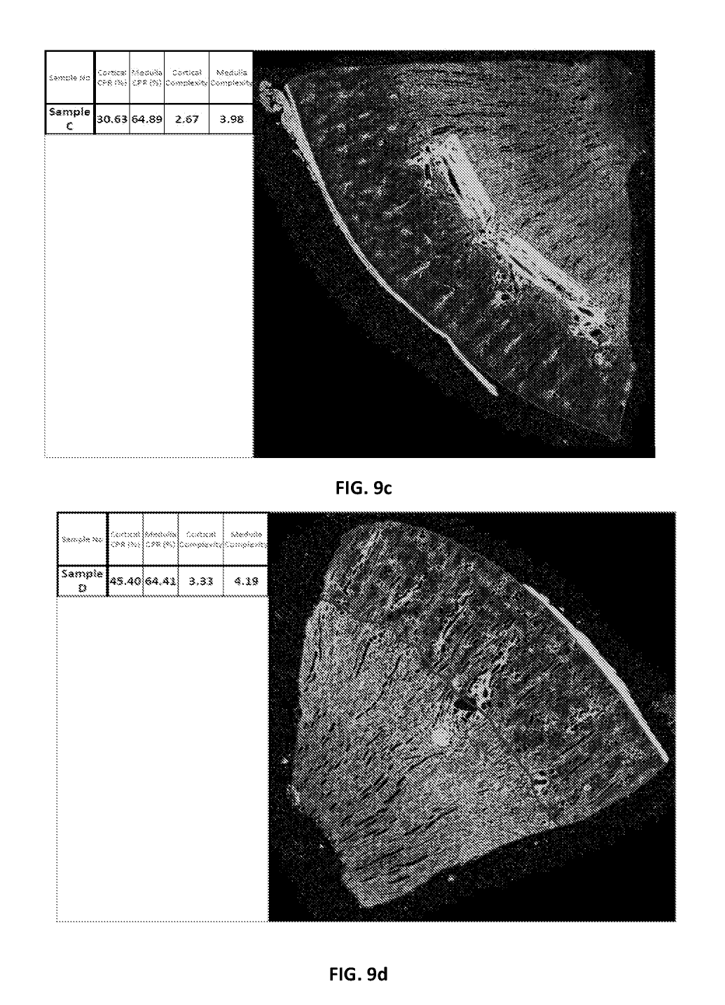

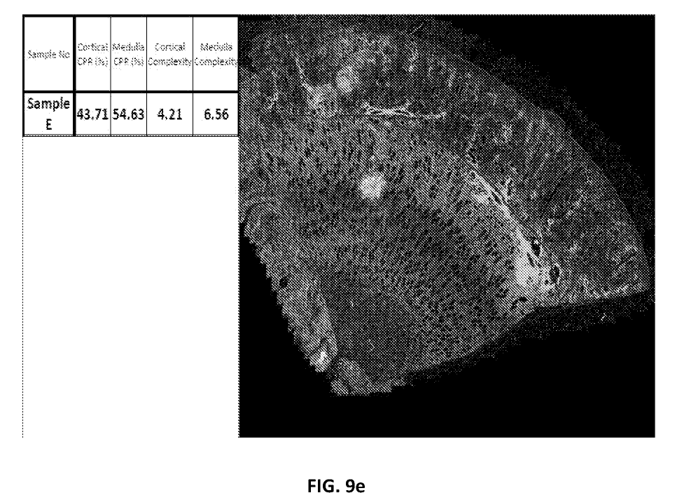

Male CD-1 mice weighing 25-35 g (6-8 week) were housed in the facility according to ethical and legal guidelines in a temperature and light controlled environment. For the duration of the experiment mice were maintained on a low sodium diet. Cyclosporine A (CsA) was made up as a 1 mg/ml stock solution in olive oil. CsA was administered by intraperitoneal injection (15 mg/kg/day), daily for 1 week or 4 weeks. The 0 week CsA group was utilised to establish baseline of the study about toxic effects of CsA before overt histological alterations had manifested. Mice had free access to food and water throughout the experiments. Across the 4 weeks study conducted, mice were sacrificed at predetermined time points and kidneys were collected for histological analysis using the method and system of the present disclosure. Particularly, part of kidney was snap frozen in liquid N₂ and part was fixed in neutral buffered formalin to prepare slide free from staining. Progression of fibrosis in the kidney sampled from 0 days, 7 days, 14 days, 21 days, and 28 days are respectively illustrated in FIG. 9(a)-(e). At 0 days, the kidney showed no fibrosis in both in cortical and medulla area. By 7 days, fibrosis and deposition of collagen became apparent in medulla area adjacent to the cortical. The fibrosis advanced deeper into medulla area by days 14 with light deposition of collagen in the cortical area too. The kidneys sampled were abundantly filled with collagen fibers by the 21 and 28 days as shown in FIGS. 9(d) and 9(e). The disclosed system employed in the study featured pattern recognition capability to distinguish medulla and cortical area in the kidney and consequently computed absolute TAC for each recognized area as shown in FIG. 9(a)-(e). Further, simplified illustrations are provided in FIGS. 8a and 8b to show one possible way to derive absolute TCA or TCQ in the present disclosure.

It is to be understood that the present invention may be embodied in other specific forms and is not limited to the sole embodiment described above. However modification and equivalents of the disclosed concepts such as those which readily occur to one skilled in the art are intended to be included within the scope of the claims which are appended thereto.

1. A method of image generating and analyzing for quantifying collagen fibers in a biological test specimen comprising:
irradiating an interested region of the test specimen using an electromagnetic light source through an optical assembly at an excitation wavelength, the irradiated test specimen having excitable materials and optically non-linear materials in the collagen fibers respectively leading to concurrent emission of a first electromagnetic signal caused by two-photon excitation (TPE) and a second electromagnetic signal as a result of second harmonic generation (SHG);
manipulating the first and second electromagnetic signals emitted from the test specimen through the optical assembly;
recording the manipulated first and the second electromagnetic signals through one or more sensors;
using the concurrently recorded first and second electromagnetic signals to generate a co-localized image having a set of image properties and being indicative of spatial distribution of the collagen fibers in the interested region; and
quantifying one or more scalar features and distribution features of the co-localized image to generate quantified result for each scalar and/or distribution feature, and deposition of collagen fibers in the test specimen using the quantified results for the extracted scalar and distribution features.

2. The method of claim 1, wherein the distribution features of the co-localized image are any one or combinations of intensity of the second electromagnetic signal, distribution of the second electromagnetic signal, gray level co-occurrence matrix (GLCM), thickness of collagen fiber, length of the collagen fibers, orientation of the collagen fibers, and straightness of the collagen fibers.

3. The method of claim 1, wherein the scalar features of the co-localized image are any one or combinations of collagen proportion ratio, total average collagen, complexity of collagen fibers, and fragment average ratio of the collagen fibers.

4. The method of claim 2 further comprising the step of calculating at least one of mean, variance, skewness, kurtosis, energy and entropy for each of the distribution features.

5. The method of claim 3, further comprising the step of deriving an absolute total average collagen by way of subjecting the co-localized image to multiple iterations of enhancement that each iteration of enhancement corresponds to a quotient of enhancement including N-fold amplification of the default pixel intensity of the co-localized image, calculating total average collagen for each iteration, generating a graft with the calculated total average collagen plotting against the quotient of enhancement, identifying a inflexion point from the plotted graph, and referring the total average collagen on the graph corresponding to the identified inflexion point to derive the absolute total average collagen, wherein N is 0.1 to 100.

6. The method of claim 5 further comprising the step of identifying collagen fibers associated to blood vessels of the test specimen generated in the co-localized image and excluding the identified collagen fibers associated to blood vessels in deriving the absolute total average collagen.

7. The method of claim 5 further comprising the step of mapping the generated graph against a standard graph calculated from a non-diseased specimen and deriving a prognosis towards a diseased state associated to the test specimen based upon relative distance between the mapped generated graph and the standard graph, wherein the generated and standard graphs are line graph.

8. The method of claim 5 further comprising the step of deriving a prognosis towards a diseased state associated to the test specimen based upon a distance of the inflexion point of the generated graph in relation to a standard graph calculated from a non-diseased specimen.

9. The method of claim 1, wherein the excitation wavelength is 700 to 850 nm.

10. The method of claim 1, wherein the biological test specimen is trimmed to a thickness of 1 to 5 μm and free from any staining.

11. An image generating and analyzing system for quantifying presence of collagen fibers in a biological test specimen comprising:
a platform for deposition of the test specimen thereon;
an electromagnetic light source coupling to the platform to direct a radiation at an excitation wavelength to an interested region of the test specimen, the radiation resulting in emission of a first and a second electromagnetic signals respectively caused by two-photon excitation (TPE) of excitable material and second harmonic generation (SHG) of optically non-linear materials in the collagen fibers in the test specimen;
a first sensor and a second sensor being arranged to respectively real-time record the emitted first and second electromagnetic signals and convert the received first and second electromagnetic signals separately into a first digital signal and a second digital signal;
an optical assembly for manipulating the radiation directed to the test specimen and the emitted first and second electromagnetic signals prior to having the first and second electromagnetic signals recorded by the first and second sensors;
a microprocessor unit being configured to use the first and second electromagnetic signals in generating a co-localized image having a set of image properties and being indicative of spatial distribution of the collagen fibers in the interested region, quantify one or more scalar features and distribution features of the co-localized image to generate quantified result for each scalar and/or distribution feature, quantifying collagen deposition in the test specimen using the quantified results for the extracted scalar and distribution features; and
a visual display, in communication with the microprocessor unit, providing an interface comprising one or more modules for customizing the set of adjustable parameters, displaying the co-localized image, quantified results of the scalar features and distribution features, and quantified collagen deposition.

12. The system of claim 11, wherein the distribution features of the co-localized image are any one or combinations of intensity and/or distribution of the second electromagnetic signal, intensity and/or distribution of the first electromagnetic signal, gray level co-occurrence matrix (GLCM), thickness of collagen fiber, length of the collagen fibers, orientation of the collagen fibers, and straightness of the collagen fibers.

13. The system of claim 11, wherein the scalar features of the co-localized image are any one or combinations of collagen proportion ratio, total average collagen, complexity of collagen fibers, and fragment average ratio of the collagen fibers.

14. The system of claim 12, wherein the microprocessor unit is configured to compute at least one of mean, variance, skewness, kurtosis, energy and entropy for each of the distribution features.

15. The system of claim 13, wherein the microprocessor unit is configured to compute an absolute total average collagen by way of subjecting the co-localized image to multiple iterations of enhancement that each iteration of enhancement corresponds to a quotient of enhancement including N-fold amplification of the default pixel intensity of the co-localized image, computing total average collagen for each iteration, generating a graft with the computed total average collagen plotting against the quotient of enhancement, identifying a inflexion point from the plotted graph, and referring the total average collagen on the graph corresponding to the identified inflexion point to derive the absolute total average collagen, wherein N is 0.1 to 100.

16. The system of claim 15, wherein the microprocessor unit is configured to identify collagen fibers associated to blood vessels of the test specimen generated in the co-localized image and exclude the identified collagen fibers associated to blood vessels in deriving the absolute total average collagen.

17. The method of claim 15, wherein the microprocessor unit is configured to map the generated graph against a standard graph computed from a non-diseased specimen and derive a prognosis towards a diseased state associated to the test specimen based upon relative distance between the mapped generated graph and the standard graph, wherein the generated and standard graphs are line graph.

18. The system of claim 15, wherein the microprocessor unit is configured to derive a prognosis towards a diseased state associated to the test specimen based upon a distance of the inflexion point of the generated graph in relation to a standard graph computed from a non-diseased specimen.

19. The system of claim 11, wherein the excitation wavelength is 700 to 850 nm.

20. The system of claim 11, wherein the biological test specimen is trimmed to a thickness of 1 to 5 μm and free from any staining.
